(19) United States
(12) Patent Application Publication
(10) Pub. No.: US 2026/0125746 A1
(43) Pub. Date: May 7, 2026

(54) METHODS AND COMPOSITIONS FOR SUBSTRATE SURFACE CHEMISTRY

(71) Applicant: esBiolab, LLC, San Diego, CA (US)

(72) Inventors: Aijie Han, San Diego, CA (US); Wei Li, San Diego, CA (US); Liangliang Qiang, San Diego, CA (US)

(21) Appl. No.: 19/117,918

(22) PCT Filed: Oct. 4, 2023
(86) PCT No.: PCT/US2023/075918

§ 371 Date: Apr. 2, 2025

Related U.S. Application Data
(60) Provisional application No. 63/413,462, filed on Oct. 5, 2022.

(51) Int. Cl. C12Q 1/6834; C12Q 1/6816; C12Q 1/6874

(52) U.S. Cl. CPC C12Q 1/6834; C12Q 1/6816; C12Q 1/6874

(57) ABSTRACT

The present disclosure relates in some aspects to methods and compositions for generating modified surfaces for microfluidic device (e.g., flow cells) and for characterizing the surface chemistry of the modified surfaces, such as the relative density of biomolecules immobilized on the surfaces.

### CROSS-REFERENCE TO RELATED APPLICATION

This application claims the priority benefit of U.S. Provisional Patent Application Ser. No. 63/413,462, filed Oct. 5, 2022, the contents of which are incorporated herein by reference in its entirety.

### FIELD

The present disclosure relates in some aspects to methods and compositions for manufacturing substrates (e.g., in microfluidic devices such as flow cells) having surface modifications, and/or for characterizing surface chemistry of manufactured substrates. Methods of using the manufactured and/or characterized substrates are also disclosed, e.g., for nucleic acid analysis including DNA sequencing.

### BACKGROUND

Microfluidic devices with surface treatment, e.g., surface immobilized DNA primers, antibodies and peptides, etc. are useful in biomolecular assay, DNA sequencing, protein sequencing and other biomolecular target detections. For instance, the density and/or quality of immobilized molecules on the surface of microfluidic device need to be characterized or measured to guarantee consistent results for downstream applications such as DNA clustering and sequencing, protein/antibody quantification, and DNA PCR reaction. However, existing methods for manufacturing modified surfaces and characterizing surface chemistry can be time consuming and expensive and can suffer from low accuracy. There remains a need for improved microfluidic surface treatment and characterization methods. The present disclosure addresses such and other needs.

### SUMMARY

The most common way to measure the density of surface molecules (e.g., DNA oligos) is by hybridization with complementary DNA oligo probes with detectable tags such as fluorescent dyes. However, this process is very time consuming, and the accuracy of measurement can be affected by the sequence of probe, salinity, pH, and/or temperature and concentration of probes. In some embodiments, provided herein are compositions and methods for measuring the surface density of molecules on a substrate (e.g., a flow cell) in a fast, accurate, and cost-effective manner. In some embodiments, the methods provided herein can achieve fast and more accurate measurements of surface immobilized biomolecule density with more than 40% cost reduction.

### BRIEF DESCRIPTION OF THE DRAWINGS

The drawings illustrate certain features and advantages of this disclosure. These embodiments are not intended to limit the scope of the appended claims in any manner.

FIG. 1 shows a schematic for characterization of surface molecular density by hybridization with fluorescent DNA oligo probes.

FIG. 2 shows a schematic for characterization of surface molecular density using internal reference oligos with fluorescent tags.

FIGS. 3A-3B show fluorescent counts of surface chemistry with different amounts oligo-dye added. FIG. 3A shows fluorescent signal intensities measured from internal immobilized oligo-dye. FIG. 3B shows fluorescent signal intensities measured by hybridization with an oligo-dye.

FIG. 4 shows the correlation of the fluorescent counts using the hybridized probe method and the internal dye for surfaces with different immobilized molecular densities.

FIG. 5 compares workflows for measuring oligo primer density on a flow chip surface. FIG. 5A shows a flowchart for the hybridization-based method. FIG. 5B shows a flowchart for the internal oligo-dye method.

FIG. 6 shows representative images of fluorescent probe staining of flow chip surfaces. FIG. 6A shows non-uniform staining of fluorescent probes on flow chip surface under low flow rate. FIG. 6B shows uniform staining of fluorescent probes on flow chip surface.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

Biological arrays, ordered or random, can be used to detect and analyze molecules, including DNA and RNA. In these applications, the arrays can be engineered to include probes for nucleotide sequences present in genes of humans and other organisms. In certain applications, for example, individual DNA and RNA probes may be attached at locations on a substrate, randomly or in a geometric grid (e.g., on a substrate with pre-patterned micro-structures, such as depressions/wells).

Biological arrays may be used for nucleic acid sequencing. In general, genetic sequencing involves determining the order of nucleotides or nucleic acids in a length of genetic material, such as a fragment of DNA or RNA. Increasingly longer sequences of base pairs are being analyzed, and the resulting sequence information may be used in various bioinformatics methods to logically fit fragments together so as to reliably determine the sequence of extensive lengths of genetic material from which the fragments were derived. Automated, computer-based examination of characteristic fragments have been developed, and have been used in genome mapping, identification of genes and their function, evaluation of risks of certain conditions and disease states, and so forth. Beyond these applications, biological arrays may be used for the detection and evaluation of a wide range of molecules, families of molecules, genetic expression levels, single nucleotide polymorphisms, and genotyping.

In some aspects, provided herein are methods to improve quality control of surface grafted molecular with simplified process and better uniformity or accuracy than hybridization-based method.

In some embodiments, provided herein is a method, comprising: a) contacting a substrate with a plurality of oligonucleotide molecules, wherein: the plurality of oligonucleotide molecules comprise detectably labeled oligonucleotide molecules that count for no more than 90% of the plurality of oligonucleotide molecules; b) allowing oligonucleotide molecule immobilization on a surface of the substrate; c) separating oligonucleotide molecules that are not immobilized on the surface from the substrate; and d) detecting signals associated with detectably labeled oligonucleotide molecules among oligonucleotide molecules that are immobilized on the surface. In some embodiments, the substrate is a solid substrate. In some embodiments, the substrate is a planar or a curved substrate, optionally wherein the substrate is a flow chip, a slide, a cover slip, or a microarray substrate.

In any of the embodiments herein, the substrate may but does not need to comprise a micro-pattern on the surface prior to the oligonucleotide molecule immobilization. In any of the embodiments herein, the micro-pattern can comprise one or more depressions and/or one or more protrusions on the surface, optionally wherein the micro-pattern comprises wells, spots, pads, posts, stripes, swirls, lines, triangles, rectangles, circles, arcs, checks, plaids, diagonals, arrows, squares, and/or cross-hatches. In any of the embodiments herein, the micro-pattern can comprise nanowells. In any of the embodiments herein, the substrate may but does not need to be a nanowells array.

In any of the embodiments herein, the substrate may but does not need to comprise biomolecules immobilized on the surface prior to the oligonucleotide molecule immobilization. In some embodiments, the biomolecule can comprise a nucleic acid, a polypeptide, a carbohydrate, or a lipid.

In any of the embodiments herein, the substrate may be pretreated prior to the oligonucleotide molecule immobilization, optionally wherein the substrate is pretreated with a silane, optionally wherein the substrate is pretreated with (3-Aminopropyl)triethoxysilane or triethoxy 2-(7-oxabicyclo-heptan-3-yl)ethyl silane. In any of the embodiments herein, the substrate may comprise a polymer on the surface, optionally wherein the polymer is a hydrogel.

In any of the embodiments herein, the detectably labeled oligonucleotide molecules may account for no more than 50% of the plurality of oligonucleotide molecules. In any of the embodiments herein, the detectably labeled oligonucleotide molecules may account for no more than 10% of the plurality of oligonucleotide molecules. In any of the embodiments herein, the detectably labeled oligonucleotide molecules may account for no more than 5% of the plurality of oligonucleotide molecules. In any of the embodiments herein, the detectably labeled oligonucleotide molecules may account for no more than 3% of the plurality of oligonucleotide molecules. In any of the embodiments herein, the detectably labeled oligonucleotide molecules may account for no more than 2% of the plurality of oligonucleotide molecules. In any of the embodiments herein, the detectably labeled oligonucleotide molecules may account for no more than 1% of the plurality of oligonucleotide molecules. In any of the embodiments herein, the detectably labeled oligonucleotide molecules may account for no more than 0.5% of the plurality of oligonucleotide molecules.

In any of the embodiments herein, the plurality of oligonucleotide molecules may have the same nucleic acid sequence, or may comprise two or more oligonucleotide molecules having different nucleic acid sequences. In any of the embodiments herein, the plurality of oligonucleotide molecules may be between about 10 and about 100 nucleotides in length, optionally between about 15 and about 75 nucleotides in length, or optionally between about 20 and about 50 nucleotides in length.

In any of the embodiments herein, the plurality of oligonucleotide molecules may comprise partial or full-length primer sequences. In any of the embodiments herein, for each detectably labeled oligonucleotide molecule, the detectable label can be at the 3′ end, 5′end, or an internal nucleic acid residue of the oligonucleotide molecule.

In any of the embodiments herein, for each detectably labeled oligonucleotide molecule, the detectable label and the oligonucleotide molecule can be directly conjugated to each other, or indirectly conjugated to each other via a spacer, optionally wherein the spacer is a cleavable or degradable spacer. In any of the embodiments herein, for each detectably labeled oligonucleotide molecule, the detectable label and the oligonucleotide molecule can be covalently or non-covalently conjugated to each other. In any of the embodiments herein, for each detectably labeled oligonucleotide molecule, the detectable label and the oligonucleotide molecule can be dissociable from each other.

In any of the embodiments herein, the detectable label can be covalently conjugated to the oligonucleotide molecule, and the detectable label can be chemically or enzymatically cleavable. In any of the embodiments herein, the detectable label can be covalently conjugated to a uracil (U) residue which is enzymatically cleavable.

In any of the embodiments herein, the detectable label can be non-covalently conjugated to the oligonucleotide molecule, and the detectable label is chemically or enzymatically cleavable. In any of the embodiments herein, the detectable label can be coupled to a binder and the oligonucleotide molecule can be coupled to a binding partner of the binder, and wherein the binder and the binding partner can form a non-covalent binding pair. In any of the embodiments herein, the binder can comprise a biotin or an analog or variant therefore, and the binding partner can comprise a streptavidin, an avidin, or an analog or variant therefore, optionally wherein the biotin analog is desthiobiotin. In any of the embodiments herein, the binding between the desthiobiotin-coupled detectable label and the streptavidin- or avidin-coupled oligonucleotide molecule can be dissociable using biotin that competes with desthiobiotin for binding to streptavidin or avidin.

In any of the embodiments herein, each detectably labeled oligonucleotide molecule, independently of one another, may comprise one, two, three, or more molecules of the same detectably label or different detectably labels. In any of the embodiments herein, the detectable label can be a fluorescent label. In any of the embodiments herein, the detectable label can be an Atto dye, optionally wherein the fluorescent label is Atto 532, Alexa Fluor 532, Cy3, or Cy5.

In any of the embodiments herein, the oligonucleotide molecules can be directly immobilized on the surface, or indirectly immobilized on the surface via a linker. In any of the embodiments herein, the oligonucleotide molecules can be covalently or non-covalently immobilized on the surface. In any of the embodiments herein, the oligonucleotide molecules can be randomly immobilized on the surface, or can be immobilized at ordered locations on the surface. In any of the embodiments herein, the 5′ ends of the oligonucleotide molecules can be immobilized on the surface. In any of the embodiments herein, the 3′ end of the immobilized oligonucleotide molecules which are not detectably labeled can comprise a hydroxyl group.

In any of the embodiments herein, the separating step may comprise washing the surface of the substrate. In any of the embodiments herein, the detecting step may comprise imaging the surface of the substrate. In any of the embodiments herein, the imaging can be performed using an imaging buffer. In any of the embodiments herein, the method may comprise generating a relative density of immobilized oligonucleotide molecules on the surface of the substrate. In any of the embodiments herein, the method may comprise storing the substrate with immobilized oligonucleotide molecules in a storage buffer. In any of the embodiments herein, the storage buffer may be the same as the imaging buffer.

In any of the embodiments herein, the method may but does not need to comprise removing detectable labels from the immobilized detectably labeled probes after the detecting step. In any of the embodiments herein, the method may but does not need to comprise hybridizing a nucleic acid probe to the immobilized oligonucleotide molecules on the surface of the substrate before the detecting step.

In any of the embodiments herein, the method may comprise applying a solution comprising the plurality of oligonucleotide molecules on the surface, or dipping the substrate in a solution comprising the plurality of oligonucleotide molecules.

In any of the embodiments herein, the method may comprise using the oligonucleotide molecules immobilized on the surface as primers for hybridizing to complementary primer sequences in nucleic acid molecules to be amplified. In any of the embodiments herein, the method may comprise extending the oligonucleotide molecules immobilized on the surface using the nucleic acid molecules hybridized thereto as templates for primer extension by a polymerase, thereby generating complementary copies of the nucleic acid molecules. In any of the embodiments herein, the method may comprise generating a cluster of amplified nucleic acid molecules of each nucleic acid molecule on the surface of the substrate. In any of the embodiments herein, the method may comprise performing sequencing-by-synthesis (SBS) cycles on the surface, thereby determining at least a portion of the sequences of the nucleic acid molecules.

In some embodiments, disclosed herein is a method, comprising: a) contacting a flow cell substrate with a plurality of oligonucleotide molecules comprising no more than 10% of fluorescently labeled oligonucleotide molecules; b) allowing oligonucleotide molecule immobilization on a surface of the flow cell substrate; c) separating oligonucleotide molecules that are not immobilized on the surface from the flow cell substrate; d) imaging the surface in an imaging buffer to detect the fluorescently labels on the surface, thereby detecting a relatively density of oligonucleotide molecules that are immobilized on the flow cell substrate.

In any of the embodiments herein, the plurality of oligonucleotide molecules may comprise no more than 5% of fluorescently labeled oligonucleotide molecules. In any of the embodiments herein, the plurality of oligonucleotide molecules may comprise no more than 2% of fluorescently labeled oligonucleotide molecules.

In any of the embodiments herein, the fluorescently labels of fluorescently labeled oligonucleotide molecules immobilized on the flow cell substrate may but do not need to be removed after the imaging step. In any of the embodiments herein, the method may comprise storing and/or shipping the flow cell substrate comprising immobilized oligonucleotide molecules in the imaging buffer, optionally wherein the flow cell substrate is stored and/or shipped at a temperature between about 2° C. and about 60° C., optionally between about 4° C. and room temperature. In any of the embodiments herein, the method may comprise using the immobilized oligonucleotide molecules as primers for nucleic acid clustering on the flow cell substrate. In any of the embodiments herein, the method may comprise using the flow cell substrate comprising immobilized oligonucleotide molecules for nucleic acid sequencing.

### EXAMPLES

The following examples are included for illustrative purposes only and are not intended to limit the scope of the present disclosure.

### Example 1: Measuring Molecular Surface Density Using Hybridized Fluorescent Probes

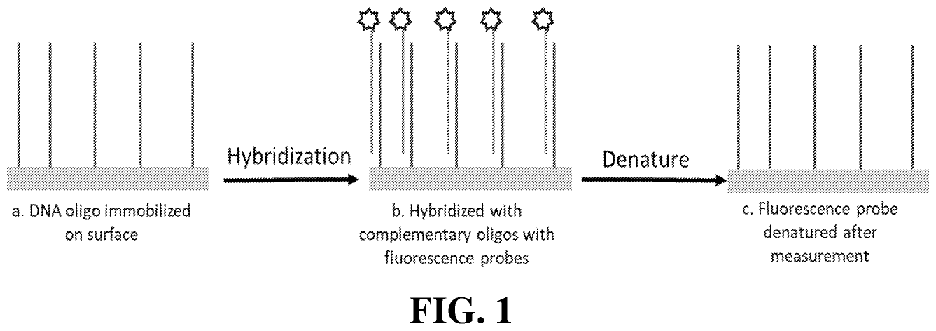

As shown in FIG. 1, in typical biochips with DNA primer grafted surface, the surface was grafted with oligos and washed with buffer to remove the excess oligo. Then the surface was incubated with complementary oligo-dye probe around the melting point of the double stranded DNA for a few minutes. The surface was washed carefully with buffer at certain temperature to remove excessive probes. The surface was scanned by fluorescence scanner to measure the intensity of the fluorescence signal from the dye on the oligo probe. The hybridized probes need to be removed after measurement by denaturation, e.g., with sodium hydroxide solution, otherwise the surface would be blocked by the probe and would not work for many downstream applications.

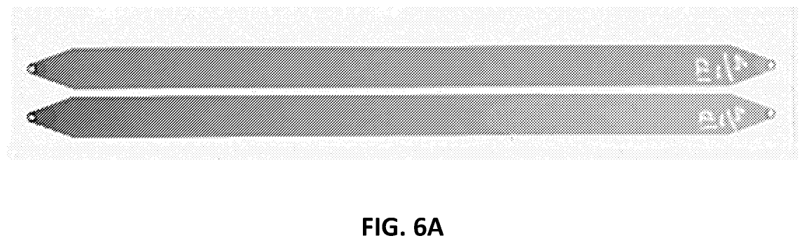

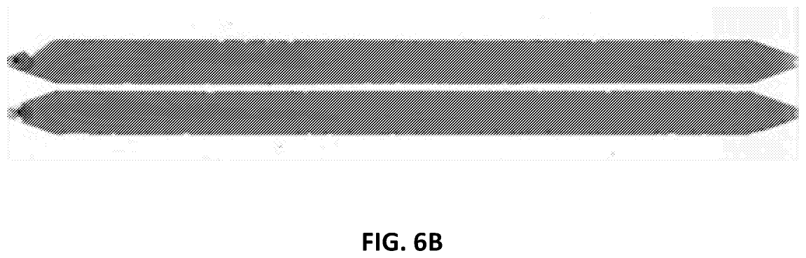

The method shown in FIG. 1 has disadvantages. For example, the hybridization condition is very critical, and the signal is highly affected by the hybridization temperature, pH, salinity, and time. The probe needs to be removed by strong basic solution or other harsh organic solvent which may damage the grafted DNA oligo on the surface. In addition, at least four separated steps (e.g., as shown in FIG. 5A) are involved to measure the relative densities of the surface oligo which is quite labor intensive and high cost. The measured fluorescent intensity may not proportion to the surface primer density at high density range due to dye self-quenching, concentration quenching or energy transfer. In addition, under low flow rate (e.g., for probe hybridization), non-uniform staining of fluorescent probes on flow chip surface has been observed, for instance, as shown in FIG. 6A, with higher staining of fluorescent probes on the flow chip surface near the inlet and lower staining near the outlet. The non-uniform staining of fluorescent probes is particularly frequent on flow chip surfaces having high surface oligo densities. Although non-uniform staining can be alleviated to some degree by increasing the flow rate (e.g., as shown in FIG. 6B), the problem remains challenging to address for flow chip surfaces having high surface oligo densities.

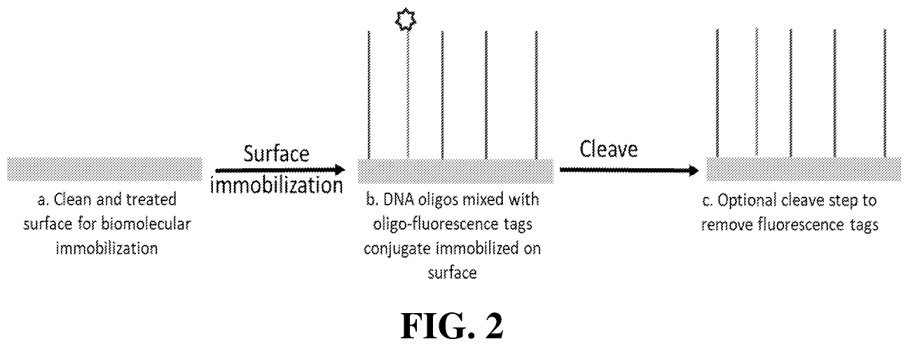

A method comprising measuring molecular surface density using fluorescent probes internal in grafted oligos is illustrated in FIG. 2. A surface was cleaned and pretreated with chemical for surface oligo grafting using the same protocol as in FIG. 1. The immobilization protocol was also same as in FIG. 1 except 0.5-10% of surface primers have fluorescence tags connected to the oligo by covalent or non-covalent bond. The linkage between the dye and oligo can be 5′, 3′ end, or internal base modification. The internal fluorescent probes (e.g., oligo-dye conjugates) was linked to the surface in the same manner, as other oligo primers that are not fluorescently labeled. To measure the surface density, the fluorescence images can be obtained after the surface grafting reaction with properly washing step. In this example, the washing buffer's pH, temperature and salinity would not affect the fluorescence measurements, especially in cases where the oligo-dye is covalently linked to the surface. The oligo-dye can stay on the surface and no cleaving or denaturation is required, although in some case, the dye can be cleaved off the oligos.

The method illustrated in FIG. 2 has at least a number of advantages.

The surface density measurement protocol is easy and fast, and no extra reagents are required. The density of the oligos can be measured any time after grafting reaction.

The fluorescent dye on the surface will not interfere with the bridge amplification in sequencing applications, for example, in cases where the dye is grafted to the internal base and the emission spectrum of the dye is out of the sequencing dye emission range, e.g., blue dye for surface QC and red, green dyes for sequencing.

In addition, the dye can be conjugated to 3′end of oligos and cleavable group can be introduced such as disulfide group or modified nucleotide which can be digested by enzymes, such as USER Enzyme, which is a mixture of Uracil DNA glycosylase (UDG) and the DNA glycosylase-lyase Endonuclease VIII. The fluorescence dyes can be employed to calibrate the optical system of sequencing instrument and can be cleaved after DNA clustering and before sequencing cycles.

The sequencing instrument can easily identify if the chip is fresh or used by detecting the fluorescent signal from the chip surface since the used chips don't bear the fluorescence dye anymore.

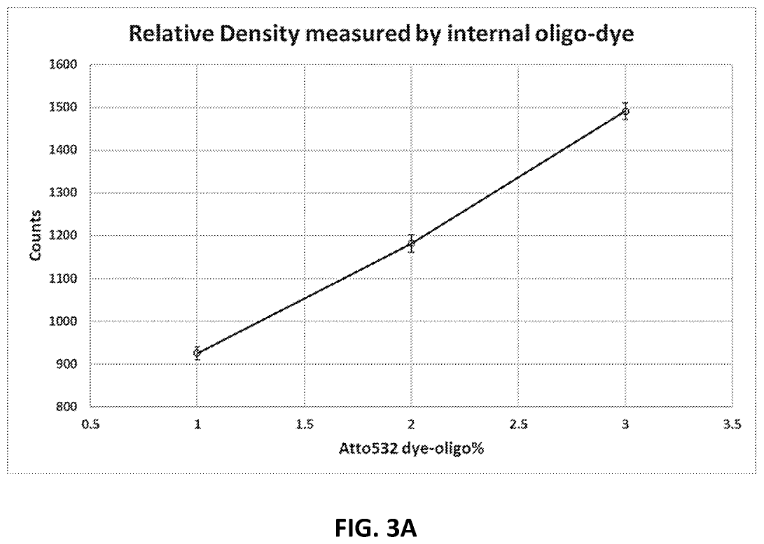

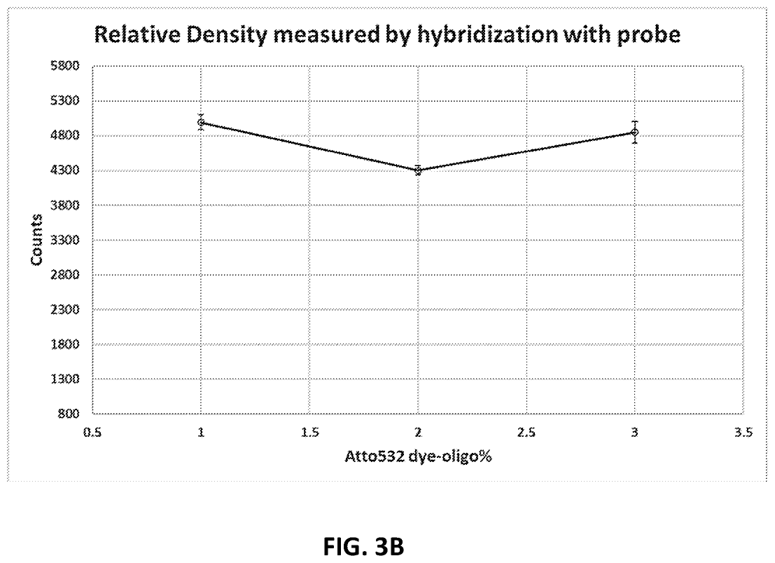

Even very low levels of internal fluorescent probes can be used for surface QC, and the level of internal fluorescent probes can be selected or adjusted based on the properties of the dye molecules, the oligos, the substrate, and/or downstream applications. For example, in FIG. 3, flow chips with different percentages of Atto 532 dye modified oligo (dye-labeled oligo molecules accounting for 1%, 2%, or 3% of all oligo molecules contacted with flow chip surface) were fabricated and fluorescence emission were measured by Typhoon scanner with green filter. All chips were also characterized by hybridization probe with Cy3 dye. All chips shown similar fluorescent intensity with hybridization probe around 4500 counts. The signals from internal grafted Atto 532 increased with higher percentage of oligo-dye on surface. To balance between the signal intensity and usable oligo without dye, 2% of Atto 532 modified oligo was chosen as the standard protocol in this example. The internal reference dye also showed better linearity at high primer density range since the surface concentration of dye is about 50 times less than hybridization-based methods (e.g., hybridization-based flow chip QC shown in FIG. 1).

Overall, the method illustrated in FIG. 2 can reduce the cost for flow chip manufacturing for more 50% due to simplified work process (e.g., as shown in FIG. 5B) and reduction of expensive dye label oligo usage.

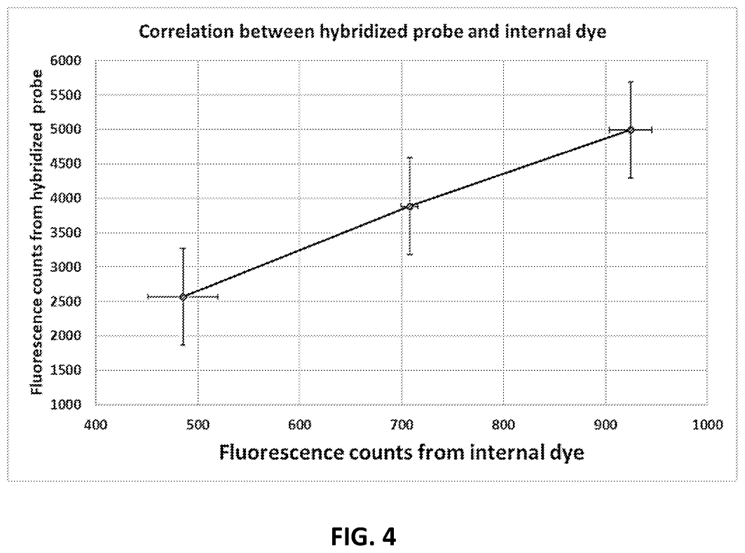

In FIG. 4, the correlation between hybridization probe and internal dye was demonstrated. The surfaces of different chips were designed to be grafted with different amounts of oligo on the surface with different reaction time. The hybridization probe showed different signal intensity according to different surface grafted oligo density. The internal dye also shown the same trend as hybridization-based method with comparable percentage standard deviation. However, the traditional hybridization-based method needs at least 8 processing steps before the flow chips are measured. In contrast, there are only two steps for density measurement with internal oligo-dye conjugates.

The internal fluorescence dye can be replaced with other biomolecules such as biotin, desthiobiotin ,2,4-Dinitrophenol and digoxigenin etc. The fluorescent dye can be introduced later with streptavidin for biotin and desthiobiotin or antibody for small hapten. The dye tag can also be released with biotin for desthiobiotin labeled oligo.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for density and/or quality characterization of surface-immobilized oligonucleotide molecules, comprising:
a) contacting a flow cell substrate with a plurality of oligonucleotide molecules, wherein: the plurality of oligonucleotide molecules comprise fluorescently labeled oligonucleotide molecules that account for no more than 10% of the plurality of oligonucleotide molecules;
b) allowing oligonucleotide molecule immobilization on a surface of the flow cell substrate;
c) separating oligonucleotide molecules that are not immobilized on the surface from the flow cell substrate; and
d) detecting signals associated with fluorescent labels of the fluorescently labeled oligonucleotide molecules among oligonucleotide molecules that are immobilized on the surface, thereby characterizing the density and/or quality of the oligonucleotide molecules immobilized on the surface of the flow cell substrate.

2-5. (canceled)

6. The method of claim 1, wherein the flow cell substrate does not comprise biomolecules immobilized on the surface prior to the oligonucleotide molecule immobilization.

7. (canceled)

8. The method of claim 1, wherein the flow cell substrate is pretreated with a silane prior to the oligonucleotide molecule immobilization.

9. The method of claim 1, wherein the flow cell substrate comprises a hydrogel on the surface.

10. (canceled)

11. The method of claim 1, wherein fluorescently labeled oligonucleotide molecules account for no more than 10% of the plurality of oligonucleotide molecules.

12. The method of claim 1, wherein fluorescently labeled oligonucleotide molecules account for no more than 5% of the plurality of oligonucleotide molecules.

13. The method of claim 1, wherein fluorescently labeled oligonucleotide molecules account for no more than 3% of the plurality of oligonucleotide molecules.

14. The method of claim 1, wherein fluorescently labeled oligonucleotide molecules account for no more than 2% of the plurality of oligonucleotide molecules.

15. The method of claim 1, wherein fluorescently labeled oligonucleotide molecules account for no more than 1% of the plurality of oligonucleotide molecules.

16. The method of claim 1, wherein fluorescently labeled oligonucleotide molecules account for no more than 0.5% of the plurality of oligonucleotide molecules.

17-19. (canceled)

20. The method of claim 1, wherein the plurality of oligonucleotide molecules comprise partial or full-length primer sequences.

21-22. (canceled)

23. The method of claim 1, wherein for each fluorescently labeled oligonucleotide molecule, the fluorescent label and the oligonucleotide molecule are covalently or non-covalently conjugated.

24. (canceled)

25. The method of claim 23, wherein the fluorescent label is covalently conjugated to the oligonucleotide molecule, and the fluorescent label is chemically or enzymatically cleavable.

26-34. (canceled)

35. The method of claim 1, wherein the oligonucleotide molecules are covalently immobilized on the surface.

36-42. (canceled)

43. The method of claim 1, comprising generating a relative density of immobilized oligonucleotide molecules on the surface of the flow cell substrate.

44. The method of claim 1, comprising storing and/shipping the flow cell substrate with immobilized oligonucleotide molecules in a storage buffer between about 4° C. and room temperature.

45-47. (canceled)

48. The method of claim 1, which does not comprise hybridizing a nucleic acid probe to the immobilized oligonucleotide molecules on the surface of the flow cell substrate before the detecting step.

49. (canceled)

50. The method of claim 1, comprising using the oligonucleotide molecules immobilized on the surface as primers for hybridizing to complementary sequences in nucleic acid molecules to be amplified.

51. (canceled)

52. The method of claim 50, comprising generating a cluster of amplified nucleic acid molecules of each nucleic acid molecule on the surface of the flow cell substrate.

53. The method of claim 52, comprising performing sequencing-by-synthesis (SBS) cycles on the surface without removing the fluorescent labels from the immobilized fluorescently labeled oligonucleotide molecules, thereby determining at least a portion of the sequences of the nucleic acid molecules.

54. The method of claim 1, wherein the detecting comprises imaging the surface in an imaging buffer to detect the fluorescent labels on the surface, thereby detecting a relative density of oligonucleotide molecules that are immobilized on the flow cell substrate.

55-57. (canceled)

58. The method of claim 54, comprising storing and/or shipping the flow cell substrate comprising immobilized oligonucleotide molecules in the imaging buffer.

59. The method of claim 54, comprising using the immobilized oligonucleotide molecules as primers for nucleic acid clustering and nucleic acid sequencing on the flow cell substrate.

60. (canceled)
